(19) United States
(12) Patent Application Publication
(10) Pub. No.: US 2019/0151399 A1
(43) Pub. Date: May 23, 2019

(54) METHODS FOR STIMULATING CONVERSION OF BODY FAT INTO MUSCLE MASS

(71) Applicant: GELITA AG, Eberbach (DE)

(72) Inventors: Stephan Hausmanns, Heidelberg (DE); Hans-Christoph Kneféli, Wiesenbach (DE); Steffen Oesser, Glucksburg (DE); Hans-Ulrich Frech, Weinheim (DE)

(21) Appl. No.: 16/257,527

(22) Filed: Jan. 25, 2019

Related U.S. Application Data
(62) Division of application No. 14/931,989, filed on Nov. 4, 2015.
Application No. 14/931,989 is a continuation of application No. PCT/EP2014/058673, filed on Apr. 29, 2014.

(30) Foreign Application Priority Data
May 13, 2013 (DE) 10 2013 104 897.8

(51) Int. Cl. A61K 38/01; A61K 38/39; A61K 9/00

(52) U.S. Cl. CPC A61K 38/014; A61K 9/0053; A61K 38/39

(57) ABSTRACT

The present invention relates to collagen hydrolysate as an active substance for treating sarcopenia, as an active substance against the degenerative loss of muscle mass and for improving muscle power, as an active substance for reducing the age-related loss of muscle mass, and as an active substance for stimulating the conversion of body fat mass to muscle mass. The invention further relates to a method for treating sarcopenia, comprising the repeated oral administration of collagen hydrolysate to a patient.

### CROSS-REFERENCE TO RELATED APPLICATIONS

This patent application is a continuation of International Application Number PCT/EP2014/058673, filed Apr. 29, 2014, which claims the benefit of German application DE 10 2013 104 897.8, filed May 13, 2013, which are each incorporated by reference.

### BACKGROUND OF THE INVENTION

The present invention relates to an active substance for treating sarcopenia.

The invention also relates to a method for treating sarcopenia.

The expression sarcopenia refers to a progressive loss of muscle mass and a reduction in muscle strength in humans, particularly with increasing age, the phenomenon often being observable from an age of approximately 50 years or more (see, e.g., Walston, Curr. Opin. Rheumatol. 2012(24) 623-627). A primary cause of sarcopenia is assumed, according to current knowledge, to be an age-related imbalance between the muscle-building (anabolic) and muscle-degenerating (catabolic) metabolic processes. In addition, muscle degeneration is clearly promoted by a lack of exercise and/or inadequate nutrition, which also occur more frequently with increasing age.

In order to counteract sarcopenia, therefore, firstly the stimulation of muscle generation through physical exertion is recommended, particularly through targeted muscle exercise or strength training, for example, with appropriate exercise devices in the context of a medical exercise therapy. A further starting point in order to promote muscle-building metabolic processes consists in a protein-rich diet or nutritional supplement with particular proteins or their hydrolysates, and there are indications in this regard that the branched-chain amino acids leucine, isoleucine and valine, in particular, play a special part (see, e.g., Leenders et al., Nutr. Rev. 2011(69) 675-689). Whey protein or soya protein, which contain a relatively high proportion of these amino acids are therefore often used as a nutritional supplement. The administration of isolated amino acids is also possible, but involves significantly higher costs.

### BRIEF SUMMARY OF THE INVENTION

The inventors have now unexpectedly discovered that collagen hydrolysate is also effective for the treatment of sarcopenia, specifically even in relatively small quantities relative to the recommended daily protein consumption.

Thus, an essential aspect of the present invention concerns collagen hydrolysate as an active substance for treating sarcopenia.

### DETAILED DESCRIPTION OF THE INVENTION

A further aspect of the invention relates to collagen hydrolysate as an active substance against the degenerative loss of muscle mass and for improving muscle strength.

A further aspect of the invention relates to collagen hydrolysate as an active substance for the reduction of the age-related loss of muscle mass.

A further aspect of the invention relates to collagen hydrolysate as an active substance for the stimulation of the conversion of body fat mass into muscle mass.

Collagen hydrolysate, which is manufactured particularly by means of enzymatic hydrolysis of collagen-containing starting materials of animal origin, consists of a mixture of peptides whose molecular weights are distributed, depending on the starting material and the manufacturing conditions, over a particular size range. The use of collagen hydrolysate as a nutritional supplement has long been known, specifically for the prevention or treatment of complaints related to the bones, the joints or the connective tissue, particularly since a stimulating effect of collagen peptides on the synthesis of the endogenous extracellular matrix in these tissue types has been shown (see, e.g., Bello et al., Curr. Med. Res. Opin. 2006 (22) 2221-2232). From the standpoint of nutrition physiology, however, collagen hydrolysate is regarded as a low-grade protein source, since its amino acid composition is highly imbalanced and it contains almost all the essential amino acids in only very small quantities, thus also the branched-chain amino acids leucine, isoleucine and valine. The essential tryptophan is not contained in collagen hydrolysate at all.

The advantageous effect of collagen hydrolysate in the treatment of sarcopenia, particularly for the building up of muscle mass and improving muscle strength, as was shown by a clinical study (see below), was not expected given this background.

According to the invention, the collagen hydrolysate is preferably to be administered orally, particularly in the form of a solution. Due to its very good solubility, the collagen hydrolysate can also be added to a variety of drinks without causing clouding. Acceptance by the user can be enhanced by the use of flavourless collagen hydrolysate.

Alternatively, the collagen hydrolysate can also be added to a solid food or luxury food, for example, a chocolate bar (as a “functional food”).

Favourably, the collagen hydrolysate is administered in a quantity of approximately 10 to approximately 20 g per day, for example, in a quantity of approximately 15 g per day. This quantity, which represents, for a body weight of 75 kg, approximately a quarter of the recommended daily protein intake (typically 0.8 g/kg body weight/day) is already sufficient to achieve a significant effect.

Sarcopenia and the symptoms associated therewith occurs increasingly with advancing age. The administration of the collagen hydrolysate takes place, therefore, particularly for patients at the age of 50 years or more, preferably 60 years or more, more preferably 65 years or more.

In order to achieve the described effect, it is particularly preferable if the collagen hydrolysate is administered in conjunction with muscle exercise. The muscle exercise can comprise any type of physical activity in which one or more muscle groups of the body are loaded, although a targeted exercise programme in the form of a repetition of pre-defined exercises, particularly on exercise machines is particularly effective, wherein almost any muscle group can be specifically exercised. It is known that immediately following a stimulation of the muscles in this manner, further enhancement of the muscle build-up can be induced by the intake of corresponding nutritional substances.

In the context of the present invention, it is therefore particularly favourable if the collagen hydrolysate is administered to a patient within two hours, preferably within one hour after said patient has performed muscle exercise.

Alternatively, it is also possible to administer the collagen hydrolysate immediately before the muscle exercise, particularly since then, due to the good absorbability, it is available to the metabolism even during the exercise. In contrast to some other proteins, collagen hydrolysate also represents no particular burden on the digestive tract.

The molecular weight of the collagen hydrolysate used can vary according to the invention over a very broad range, an upper limit being provided in that collagen hydrolysate, as distinct from denatured collagen or gelatin, has a sufficient degree of hydrolysis so that it is water-soluble at room temperature and does not gelatinise. Preferably, the collagen hydrolysate has a mean molecular weight of up to 5,000 kDa, particularly up to 3,500 kDa.

The collagen hydrolysate is favourably manufactured by means of enzymatic hydrolysis of a collagen-containing starting material. For this hydrolysis, in particular, endopeptidases and/or exopeptidases of microbial or plant origin are used.

The collagen-containing starting material is typically selected from skin or bone of vertebrates, preferably from mammals, particularly from cattle or pigs. The collagen hydrolysate can either be manufactured in a single-step method from these starting materials or via the intermediate step of gelatin wherein, in this case, both type A gelatin and type B gelatin can be used.

As mentioned above, the effect of the collagen hydrolysate observed according to the invention is remarkable particularly against the background that collagen hydrolysate does not contain many essential amino acids, particularly tryptophan, leucine, isoleucine and valine, or only in very small proportions. It is also not necessary in the context of the invention to supplement these amino acids, i.e., in a preferred embodiment of the invention, the collagen hydrolysate contains no added free amino acids.

Nevertheless, such supplementation of the collagen hydrolysate or a mixture with other components is possible in the context of the invention, in order, for example, to take account of further nutrition-physiological aspects. Aside from free amino acids, in particular (e.g. in the context of a protein-rich diet) further proteins can be mixed into the collagen hydrolysate, preferably whey protein, casein, lactalbumin or plant proteins (e.g., from wheat, soya or peas).

According to a further embodiment of the invention, vitamins and/or minerals can be added to the collagen hydrolysate in order to ensure sufficient provision with the relevant substances.

A further object of the present invention is a method for treating sarcopenia, comprising the repeated oral administration of collagen hydrolysate to a patient.

According to a further aspect of the invention, by means of this method, the degenerative loss of muscle mass is counteracted and muscle strength is improved.

According to a further aspect, by means of the method, the age-related loss of muscle mass is reduced.

According to a further aspect, by means of the method, the conversion of body fat mass into muscle mass is stimulated.

In a preferred embodiment, the method according to the invention further comprises the performance of muscle exercise by the patient before at least part of the administrations of collagen hydrolysate. It is particularly favourable if the administration of the collagen hydrolysate is repeated daily and the muscle exercise at least once weekly, preferably three times weekly.

It is particularly favourable if the collagen hydrolysate is administered in each case within two hours, preferably within one hour following a muscle exercise.

Further advantages and preferred embodiments of the method according to the invention, particularly with regard to the preferred quantity of collagen hydrolysate and the type of muscle exercise, have already been described in relation to the collagen hydrolysate according to the invention as an active substance and apply equally to the method.

Based on the double-blind, placebo-controlled clinical trial described below, which serves as an exemplary embodiment, the invention will now be set out in greater detail.

### BRIEF DESCRIPTION OF THE SEVERAL VIEWS OF THE DRAWING(S)

FIG. 1 shows a bar chart relating to the fat-free body mass and the body fat mass; and

FIG. 2 shows a histogram relating to the muscle strength and sensorimotor control.

### EXAMPLES

### 1. Selection of Subjects

The subjects were selected from among men aged over 65 years in whom, as stated by them, in the last three to four years, the muscle strength or physical capacity had significantly lessened. Pre-conditions for participation were, inter alia, that apart from the muscular weakness, no health problems existed and the subjects were able to undertake a three-month exercise programme.

Of 106 persons who were included in the narrower selection process following a telephone interview, 60 subjects were selected in whom, based on the measurement of the strength of the hand musculature with a dynamometer (Trailite from LiteExpress GmbH, Coesfeld), the existence of sarcopenia could be diagnosed. Type I sarcopenia was diagnosed if the hand strength lay more than one standard deviation below the normal value for a male reference population aged from 35 to 39 years, and type II sarcopenia was correspondingly diagnosed for a hand strength lower by more than two standard deviations.

By means of an extensive medical examination including a blood test, it was ensured that there was no chronic disease present in the subjects.

The 60 subjects of the trial were randomly distributed between two groups of 30 subjects each, i.e., a treatment group and a placebo group.

### 2. Muscle Exercise

Both groups performed an identical exercise programme, under supervision, over a period of 12 weeks with exercise carried out three times per week for 60 minutes each time on exercise machines (e.g., cable-and-pulley machines, weight bench, leg press, etc.) in order to load all the larger muscle groups specifically. The exercise programme was regularly tested for each subject and adapted to the individual capacity.

Subjects who missed more than 10% of the exercise units were excluded from the trial so that the number of subjects who successfully completed the trial and were taken into account in the evaluation was reduced to 26 in the treatment group and 27 in the placebo group.

### 3. Administration of Collagen Hydrolysate

The subjects in the treatment group took 15 g of collagen hydrolysate daily throughout the twelve-week trial period, respectively dissolved in 250 ml water. Enzymatically hydrolysed collagen from pork skin with a molecular weight in the range of 3,000 to 3,200 kDa was used. On the days with muscle exercise, the subjects were instructed to drink the solution as soon as possible and no later than one hour after the exercise unit.

Instead of collagen hydrolysate, the subjects of the placebo group were given the same quantity of silicon dioxide, also in 250 ml water. Silicon dioxide was used as it is a safe food additive, but has no influence on the metabolism.

### 4. The Investigated Parameters

Before and after each trial period, the body fat mass, the bone mass and the fat-free body mass of each subject was measured by means of dual-energy X-ray absorptiometry (DXA) using a Strator DR 2D Fan Beam (from Degen Medizintechnik, Heppenheim). Provided the body weight remains the same in each case, an increase in muscle mass can be concluded from a reduction of the fat proportion or an increase in the fat-free proportion.

As parameters for the muscle strength, the isokinetic strength of the quadriceps of the right leg was measured before and after the trial period (Con-Trex, Dübendorf, Switzerland).

The sensorimotor monitoring before and after the trial period was determined by means of a standardised one-leg stabilisation test (Posturo-med, Haider-Bioswing, Weiden). In this test, the lower the measurement value, the better is the sensorimotor control of the subject, which correlates, among other things, to muscle strength.

### 5. Results

The mean weight of the subjects remained substantially constant during the trial period (85.6 kg before the trial and 85.0 kg after the trial) and within the two groups there was no statistically significant weight change through the trial.

Both with an overall consideration of all the subjects (n=53) and also within the two groups (treatment group, n=26 and placebo group, n=27), however, there was a marked, i.e. statistically significant, increase in the fat-free body mass, the bone mass and the muscle strength, as well as a decrease in the fat mass and the sensorimotor control. This result is to be expected purely due to the strength training performed by all the subjects.

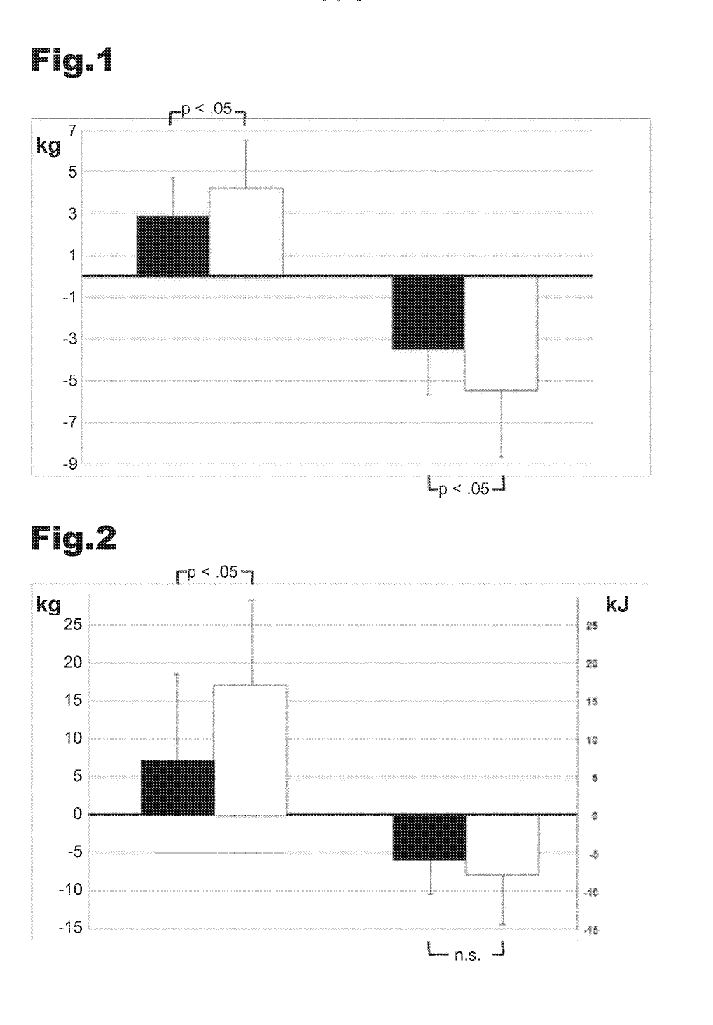

The evidence for the effectiveness of the collagen hydrolysate in the context of the present invention is found in the comparison of the measured parameters between the treatment group and the placebo group. In FIG. 1, the mean increase in fat-free body mass (left portion) and the decrease in body fat mass (right portion) is shown in kg in the form of a bar chart, respectively for the placebo group as the black bar and for the treatment group as the white bar. In FIG. 2, the mean increase in muscle strength in kg (left portion) and the mean decrease in the sensorimotor control in kJ (right portion) is shown, again as a black bar for the placebo group and as a white bar for the treatment group.

Both the increase in fat-free body mass, the decrease in the body fat mass and the increase in muscle strength are each significantly more marked for the treatment group than for the placebo group (p<0.05). The improvement in sensorimotor control is also better in the treatment group, although not to a statistically significant extent.

The trial therefore shows clearly that by means of the administration of collagen hydrolysate, a degenerative loss of muscle mass can be counteracted and an improvement in muscle strength can be achieved, and that collagen hydrolysate is suitable as an active substance for treating sarcopenia.

1-20. (canceled)

21. A method for stimulating conversion of body fat mass into muscle mass in a patient, the method comprising orally administering collagen hydrolysate to the patient repeatedly.

22. The method according to claim 21, comprising orally administering a solution of the collagen hydrolysate.

23. The method according to claim 21, comprising orally administering the collagen hydrolysate to the patient in a quantity of approximately 10 to approximately 20 g per day.

24. The method according to claim 23, comprising orally administering the collagen hydrolysate to the patient in a quantity of approximately 15 g per day.

25. The method according to claim 21, wherein the patient is aged 50 years or more.

26. The method according to claim 21, wherein the collagen hydrolysate is administered in conjunction with muscle exercise.

27. The method according to claim 26, wherein the collagen hydrolysate is administered to the patient within two hours after the patient has performed a muscle exercise.

28. The method according to claim 26, wherein the collagen hydrolysate is to be administered to the patient immediately before a muscle exercise.

29. The method according to claim 21, wherein the collagen hydrolysate has a mean molecular weight of up to 5,000 Da.

30. The method according to claim 21, wherein the collagen hydrolysate is manufactured by the enzymatic hydrolysis of a collagen-containing starting material.

31. The method according to claim 30, wherein the collagen-containing starting material is selected from skin or bone of vertebrates.

32. The method according to claim 21, wherein the patient performs a muscle exercise before at least one oral administration of collagen hydrolysate.

33. The method according to claim 32, comprising orally administering the collagen hydrolysate daily and wherein the muscle exercise is repeated at least once weekly.

34. The method according to claim 32, comprising orally administering the collagen hydrolysate to the patient within two hours after a muscle exercise.
